(19) United States
(12) Plant Patent Application Publication
(10) Pub. No.: US 2018/0098470 P1
(43) Pub. Date: Apr. 5, 2018

(54) Apple Tree Named 'Y102'

(50) ;

(71) Applicant: International Fruit Obtention (IFO) GIE, Seiches sur Ie Loir (FR)

(72) Inventor: Valérie Fouillet, Saint Sylvain d'Anjou (FR)

(73) Assignee: International Fruit Obtention (IFO) GIE, Seiches sur Ie Loir (FR)

(21) Appl. No.: 15/330,525

(22) Filed: Sep. 30, 2016

(51) Int. Cl. A01H 5/08

(52) U.S. Cl. PLT/172

(57) ABSTRACT

‘Y102’ apple tree is a new variety selected for its distinctive pink and light yellow-fleshed fruit, which is well-suited for the fresh market.

Latin name: Malus domestica.

Variety denomination: ‘Y102’.

### BACKGROUND OF THE VARIETY

‘Y102’ is a new and distinct variety of apple tree (Malus domestica) obtained from a controlled cross of ‘Golden Delicious’ (female parent, not patented)×‘SJ109’ (male parent, not patented) carried out at Seiches sur le Loir, France in 2006. Seeds obtained from the cross were planted at Seiches sur le Loir, and ‘Y102’ was selected from the resulting seedlings for propagation and further observation. ‘Y102’ was first asexually propagated by grafting in 2011 at Seiches sur le Loir, and has since been observed to remain true to type over successive asexually propagated generations.

### BRIEF DESCRIPTION OF THE VARIETY

‘Y102’ apple tree is distinguished by its fruit, which has pink flesh and is well-suited for fresh consumption. ‘Y102’ is distinguished from its parents as shown in Tables 1 and 2 below.

TABLE 1
Comparison of ‘Y102’ to ‘Golden Delicious’

| Characteristic | ‘Y102’ | ‘Golden Delicious’ |
|---|---|---|
| Harvest time | 5-10 days later than ‘Golden | Mid to late September |
| | Delicious’ | |
| Flesh Color | Pink | White |
| Skin Color | Dark orange-yellow | Yellow |

TABLE 2
Comparison of ‘Y102’ to ‘SJ109’

| Characteristic | ‘Y102’ | ‘SJ109’ |
|---|---|---|
| Fruit Shape | Round | Flat-round |
| Texture | Crunchy | Mealy, soft |
| Storageability | Moderate (2 months) | Short (less than 1 month) |
| Taste | Balanced sugar/acid | Acidic |

### BRIEF DESCRIPTION OF THE PHOTOGRAPHS

The accompanying photographs of the ‘Y102’ variety were taken in 2016 at Seiches sur le Loir, France.

FIG. 1 is a photograph of a ‘Y102’ apple tree;

FIG. 2 is a photograph of flowers and young leaves on a ‘Y102’ apple tree;

FIG. 3 is a photograph of a leaf of the ‘Y102’ apple tree; and

FIGS. 4 and 5 are photographs of whole and sectioned ‘Y102’ apples.

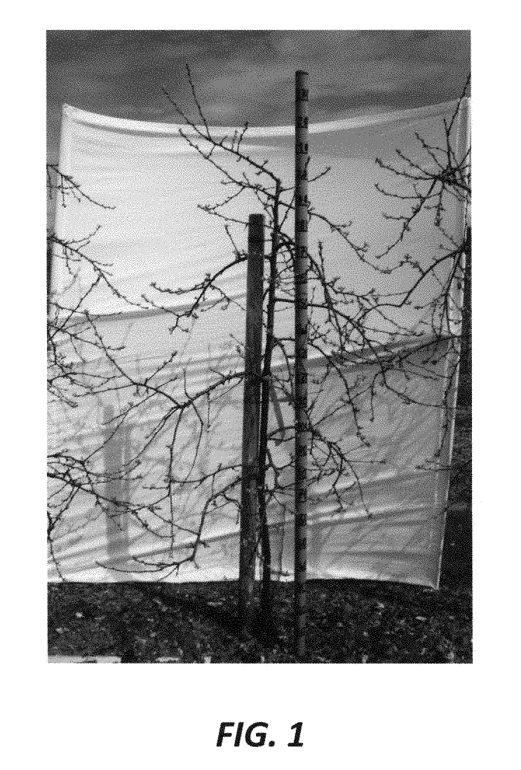

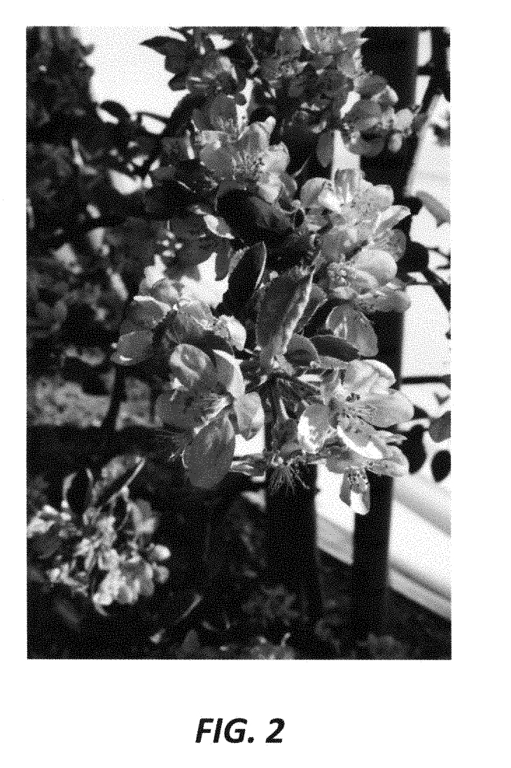

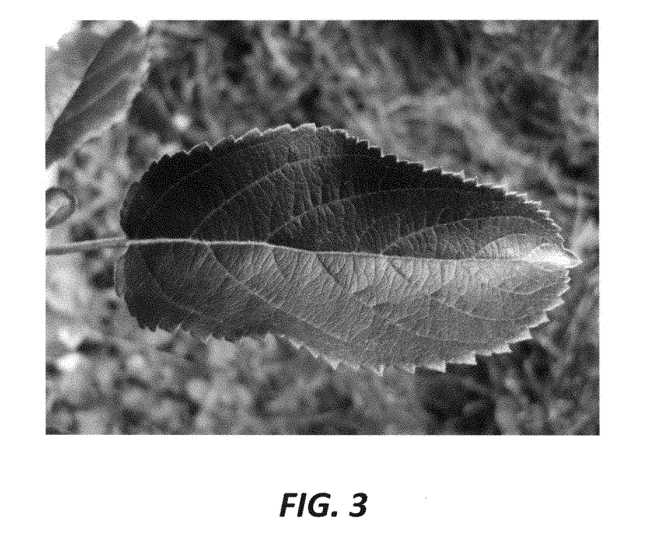

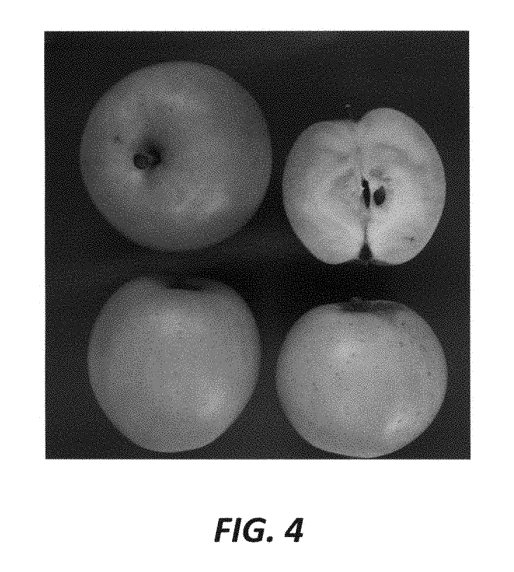

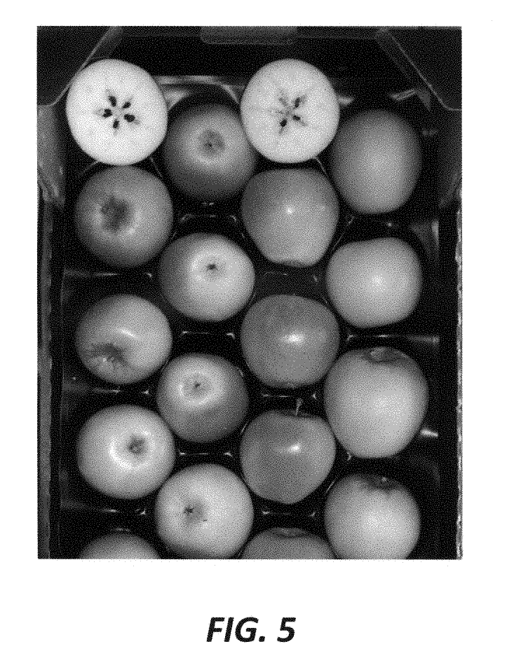

### DETAILED BOTANICAL DESCRIPTION OF THE VARIETY

The following detailed botanical description is based on observations made at Seiches sur le Loir, France during the 2016 growing season, of two year old trees grown on Pajam®2 ‘Cepiland’ rootstock (U.S. Plant Pat. No. 7,715). All colors are described according to The Royal Horticultural Society Mini Colour Chart (2005). It should be understood that the characteristics described will vary somewhat depending upon cultural practices and climatic conditions, and will vary with location and season. Quantified measurements are expressed as an average of measurements taken from a number of individual plants of the new variety. The measurements of any individual plant or any group of plants of the new variety may vary from the stated average.
Tree:
   Vigor.—Medium to high.
   Type.—Ramified.
   Habit.—Drooping.
   Height.—Approximately 2.3 m.
   Trunk diameter (at 30 cm above the graft).—34 mm.
   Bark texture.—Smooth.
   Bark coloration.—Grey N200C.
Branch (fruiting branches located at around 1 m above the graft union):
   Length.—80 cm.
   Diameter.—15 mm.
   Crotch angle.—70°.
   Bark color.—Grey N200C.
   Lenticel length.—1.2 mm.
   Lenticel color.—Dark orange yellow 22A.
   Number of lenticels per cm².—6.
One year old shoot:
   Length.—60 cm.
   Color.—Purple brown 166A.
   Pubescence.—Medium.
   Thickness.—5.5 mm to 6.5 mm.
   Internode length.—3.5 cm.
   Number of lenticels per cm².—11.
Flower buds:
   Quantity per spur.—5.
   Shape.—Ellipsoid.
   Length.—13 mm.
   Diameter.—5 mm.
   Color.—Light yellow brown 160B.
Flowers:
   Diameter of fully open flower.—30 mm.
   Relative position of petal margin.—Touching.
   Number per cluster.—5 to 7.
   Date of first bloom.—Same as ‘Cripps Pink’ (U.S. Plant Pat. No. 7,880).
   Date of full bloom.—Same as ‘Cripps Pink’.
   Pollination requirement.—‘Gala’, ‘Granny Smith’, ‘Golden Delicious’ and ‘Cripps Pink’ are suitable pollinators.
Petals:
   Number per flower.—5.
   Shape.—Elliptical.
   Length.—18 mm.
   Width.—12 mm.
   Apex.—Obtuse.
   Base.—Acute.
   Margin.—Smooth.
   Coloration of upper surface.—Pink Violet 75C.
   Coloration of lower surface.—Pink Violet 77D.
Pistils:
   Size.—11 mm.
   Color.—Light yellow green 150B.
Stigma:
   Size.—1 mm.
   Color.—Light yellow brown 163B.
Style:
   Size.—6 mm.
   Color.—Yellow green 145C.
Ovary:
   Size.—4 mm.
   Color.—Yellow green 145C.
Anthers:
   Quantity.—16 to 20.
   Size.—2 mm.
   Presence of pollen.—Yes.
   Color of pollen.—Light yellow 8C.
Pedicel:
   Length.—14 mm.
   Diameter.—1.5 mm.
   Color.—Green 139C.
Sepals:
   Quantity.—5.
   Coloration.—Green 139C.
   Shape.—Pointed.
   Margin.—Smooth.
Leaves:
   Shape.—Elliptical.
   Length.—78 mm.
   Width.—48 mm.
   Length/width ratio.—1.63.
   Blade margin.—Sharply serrate.
   Apex.—Right-angled to obtuse.
   Base shape.—Obtuse.
   Color of upper surface.—Dark green 136 A.
   Color of lower surface.—Green 137C.
   Attitude in relation to shoot.—Upwards.
Petiole:
   Length.—27 mm.
   Diameter.—1.4 mm.
   Coloration.—Yellow green 145C with some purple 58A.
Fruit:
   Quantity per cluster.—2-3.
   Diameter.—72 mm.
   Weight.—194 g.
   Ratio of height to width.—1.05.
   General shape in profile.—Globose asymmetric.
   Position of maximum diameter.—Middle of the fruit.
   Ribbing.—Absent or weak.
   Crowning at calyx end.—Weak.
   Size of eye.—Medium.
   Aperture of eye.—Weak to moderate.
   Length of sepal.—4 mm.
   Bloom of skin.—Absent or weak.
   Greasiness of skin.—Moderate.
   Amount of over color.—Moderate to large.
   Color of skin.—Orange yellow 22A.
   Intensity of over color.—Medium.
   Pattern of over color.—Only solid flush.
   Amount of russet around stalk cavity.—Weak to moderate.
   Amount of russet on cheeks.—Absent or weak.
   Area of russet around eye basin.—Absent.
   Length of stalk.—17 mm.
   Thickness of stalk.—2.5 mm.
   Depth of stalk cavity.—13 mm.
   Width of stalk cavity.—23 mm.
   Depth of eye basin.—7 mm.
   Width of eye basin.—23 mm.
   Firmness of flesh.—Medium to high.
   Flesh texture.—Fine.
   Aroma.—Medium to high.
   Juiciness.—Medium to high.
   Brix.—14.5° Brix.
   Flesh coloration.—Light red pink 38B and light yellow brown 158D.
   Stem coloration.—Brown N199C.
Seeds:
   Quantity per fruit.—8 to 9.
   Shape.—Oval — oblong.
   Coloration.—Dark brown 200 C.
Harvest:
   Time for harvest.—5-7 days after Golden.
   Number of picks.—2 to 3.
   Fruit load per tree.—High.
Disease resistance/susceptibility: No resistance noted.
Market use: Fresh eating.

I claim:

1. A new and distinct apple tree as described and illustrated herein.
